(19) United States
(12) Patent Application Publication
(10) Pub. No.: US 2010/0028305 A1
(43) Pub. Date: Feb. 4, 2010

(54) Methods for the treatment of lysosomal storage disorders

(76) Inventors: Nobuko Uchida, Palo Alto, CA (US); Yakop Jacoba, Sunnyvale, CA (US); Stanley Tamaki, Kensington, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/794,741

(22) PCT Filed: Jan. 4, 2006
(86) PCT No.: PCT/US06/00490

§ 371 Date: Sep. 22, 2009

Related U.S. Application Data
(60) Provisional application No. 60/728,440, filed on Oct. 19, 2005.
(60) Provisional application No. 60/641,517, filed on Jan. 4, 2005.

(51) Int. Cl. A61K 45/00; A61P 43/00; A61P 25/00

(52) U.S. Cl. 424/93.7

(57) ABSTRACT

Provided herein are methods for the treatment of lysosomal storage disorders characterized by a missing or defective secreted lysosomal enzyme. Such lysosomal storage disorders include, but are not limited to neuronal ceroid lipofuscinoses. The disclosed methods involve the transplantation of human multipotent neural stem cells into the CNS of patients suffering from the lysosomal storage disorder. Also provided herein are methods of reversing or slowing the progression of neurodegeneration in patients suffering from or at risk of developing neuronal ceroid lipofuscinoses.

### FIELD OF THE INVENTION

The present invention relates generally to methods for neurotransplantation of multipotent neural stem cells for the treatment of lysosomal storage disorders in which a secreted lysosomal enzyme is defective or missing.

### BACKGROUND OF THE INVENTION

Lysosomal storage disorders (“LSDs”) are the result of genetically inherited mutations in genes that code for lysosomal enzymes. The consequence of the defective or missing enzymes is the accumulation of undegraded metabolic substrates in the lysosomes that eventually lead to cell degeneration. (See Futerman and van Meer, Nature Reviews Mol. Cell Biol. 5:554-65 (2004)).

### SUMMARY OF THE INVENTION

Provided herein are methods of treating lysosomal storage disorders in mammals (e.g., in humans) by administering an effective amount of a multipotent self-renewing central nervous system neural stem cell population to the mammal. Those skilled in the art will recognize that the instant invention also encompasses the use of an effective amount of a multipotent self-renewing central nervous system neural stem cell population in the manufacture of a medicament for the treatment of a lysosomal storage disorder in a mammal (e.g., in humans).

Specifically, the lysosomal storage disorder may be a disease or disorder that is characterized by a missing or defective secreted lysosomal enzyme. Moreover, the lysosomal storage disorder may also be characterized by a mutation in a gene encoding for a secreted lysosomal enzyme. For example, the mutation may be in either the palmitoyl-protein thioesterase 1 (PPT1) gene or in the tripeptidyl peptidase I (TPP-I) gene.

Preferably, the multipotent self-renewing central nervous system neural stem cell population is obtained from a human (e.g., HuCNS-SC). The cells of the multipotent CNS neural stem cell population can be proliferated in a suspension culture or in an adherent culture prior to administration to the mammal or prior to the manufacture of the medicament.

For example, the lysosomal storage disorder to be treated in accordance with the methods and/or uses of the invention may be a neuronal ceroid lipofuscinoses. Exemplary neuronal ceroid lipofuscinoses include, but are not limited to, infantile NCL and late infantile NCL. However, those skilled in the art will recognize that other lysosomal storage disorders can also be treated using an effective amount of a multipotent self-renewing CNS stem cell population.

Moreover, in accordance with the methods of the instant invention, the effective amount of the multipotent self-renewing CNS neural stem cell population and/or the medicament for treating a lysosomal storage disorder is transplanted (or otherwise administered, injected, and/or inserted) into the CNS of the mammal. In some preferred embodiments, the mammal is a human. For example, the cells (or the medicament) may be transplanted into the hippocampus. Alternatively (or additionally), the cells (or the medicament) may be transplanted into the cortex. Those skilled in the art will recognize that the multipotent self-renewing CNS neural stem cell population and/or the medicament for treating a lysosomal storage disorder in a mammal can be transplanted into any other suitable locations within the CNS of the mammal. Determination of the appropriate CNS transplantation region suitable for treatment of a given lysosomal storage disorder is within the routine level of skill in the art.

Any suitable transplantation or administration method known to those skilled in the art can be used to administer the effective amount of the multipotent self-renewing central nervous system (CNS) neural stem cells and/or the medicament for treating a lysosomal storage disorder to the mammal in accordance with the instant methods. By way of non-limiting example, transplantation may be achieved by subcortical injection, by intraventricular injection, or by any neurotransplantation protocols known to those skilled in the art. (See, e.g., U.S. Pat. No. 6,497,872, incorporated herein by reference.)

In accordance with the methods described herein, a range of between about 3×10⁶ to about 1×10¹⁰ cells, for example between about 5×10⁸ to about 2×10⁹ cells or between about 1×10⁸ and about 5×10⁹ cells, can be administered to the mammal. For example, in one embodiment, a low dose of 5×10⁸ cells can be transplanted or implanted or injected or administered to the mammal. In another embodiment, a high dose of 1×10⁹ cells can be transplanted or implanted or injected or administered to the mammal. Those skilled in the art will recognize that the effective amount of the multipotent CNS neural stem cell population used to treat the lysosomal storage disorder can be administered either in one dose or in multiple doses.

Similarly, the medicament for treating a lysosomal storage disorder in a mammal may contain a range of between about 3×10⁶ to about 1×10¹⁰ cells, for example between about 5×10⁸ to about 2×10⁹ cells or between about 1×10⁸ to about 5×10⁹ cells. For example, in one embodiment, the medicament may contain a low dose of 5×10⁸ cells. In another embodiment, the medicament may contain a high dose of 1×10⁹ cells. Those skilled in the art will recognize that the medicament for the treatment of a lysosomal storage disorder can be administered either in one dose or in multiple doses.

Moreover, in various embodiments of the invention, the effective amount of the multipotent CNS neural stem cell population is obtained from the mammal's own neural tissue. Additionally, in other embodiments of the invention, the multipotent CNS neural stem cell population may be derived from neonatal, juvenile, or adult mammalian neural tissue.

In one specific embodiment, the instant invention also pertains to a method of treating a neuronal ceroid lipofuscinoses such as infantile or late infantile neuronal ceroid lipofuscinoses by administering a dose of between about 5×10⁸ to about 1×10⁹ multipotent self-renewing CNS neural stem cells to a subject in need thereof. In another specific embodiment, the instant invention also pertains to the use of between about 5×10⁸ to about 1×10⁹ multipotent self-renewing CNS neural stem cells in the manufacture of a medicament for the treatment of a neuronal ceroid lipofuscinoses such as infantile or late infantile neuronal ceroid lipofuscinoses in a subject.

Also provided herein are methods of reversing or slowing neurodegeneration (i.e., neuroprotection methods) in a patient suffering from or at risk for developing a lysosomal storage disorder (e.g. a neuronal ceroid lipofuscinoses) by transplanting an effective amount of a multipotent self-renewing CNS neural stem cell population into the hippocampus and/or the cortex of the patient. For example, these methods of reversing or slowing neurodegeneration can be applied to patients suffering from or at risk for developing infantile NCL or late infantile NCL.

A range of between about 3×10⁶ to about 1×10¹⁰ cells, e.g., between about 5×10⁸ to 2×10⁹ cells or about 1×10⁸ to about 5×10⁹ cells can be administered to the patient. In various embodiments, the effective amount of the multipotent self-renewing CNS neural stem cell population that is transplanted in accordance with these neuroprotection methods is 5×10⁸ cells (low dose) or 1×10⁹ cells (high dose). Those skilled in the art will recognize that the effective amount of the multipotent self-renewing CNS neural stem cell population can be transplanted in one dose or in multiple doses. Moreover, those skilled in the art will also recognize that the transplanting can occur by subcortical injection or by intraventricular injection. However, any other suitable transplantation methods known to those skilled in the art can also be employed in accordance with the claimed neuroprotection methods.

In some neuroprotection methods, the multipotent CNS neural stem cell population is obtained from the mammal's own neural tissue. Moreover, the multipotent CNS neural stem cell population can also be derived from neonatal, juvenile, or adult mammalian neural tissue.

Those skilled in the art will recognize that the instant invention also encompasses the use of an effective amount of a multipotent self-renewing CNS neural stem cell population in the manufacture of a medicament for reversing or slowing neurodegeneration in a patient suffering from or at risk for developing a lysosomal storage disorder. For example, the lysosomal storage disorder may be a neuronal ceroid lipofuscinoses, including, but not limited to, infantile NCL or late infantile NCL. Such medicaments are suitable for administration and/or transplantation into the hippocampus and/or the cortex of the patient suffering from or at risk for developing the lysosomal storage disorder.

The medicament for reversing or slowing neurodegeneration in a patient suffering from or at risk for developing a lysosomal storage disorder may contain between about 3×10⁶ to about 1×10¹⁰ cells, e.g., between about 5×10⁸ to 2×10⁹ cells or between about 1×10⁸ to about 5×10⁹ cells. In various embodiments, medicament for reversing or slowing neurodegeneration in a patient suffering from or at risk for developing a lysosomal storage disorder contains 5×10⁸ cells (low dose) or 1×10⁹ cells (high dose). Those skilled in the art will recognize that the medicament can be administered or transplanted into the host in one dose or in multiple doses. Moreover, those skilled in the art will also recognize that the medicament is suitable for transplantation or administration by subcortical injection or by intraventricular injection. However, any other suitable transplantation or administration methods known to those skilled in the art can also be employed.

The effective amount of the multipotent CNS neural stem cell population in the medicament can be obtained from the mammal's own neural tissue or it can be derived from neonatal, juvenile, or adult mammalian neural tissue.

Also provided are pharmaceutical compositions for treating lysosomal storage disorders. Such compositions may contain between about 3×10⁶ and about 1×10¹⁰ cells or between about 1×10⁸ and 5×10⁹ cells and a pharmaceutically acceptable carrier or carriers.

Any pharmaceutically carriers known to those skilled in the art can be used. In addition, the invention also provides kits containing, in one or more containers, the pharmaceutical compositions of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a graph that shows that the PPT1 & TPP1 enzymes secreted by hCNS-SC are internalized by mutant fibroblast via the mannose 6-phosphate receptor. Co-cultures in transwell plates were carried out for 7 days. Extracellular uptake can be blocked by addition of free mannose-6-phosphate to cultures. Intracellular enzyme activity of mutant fibroblasts alone (left hand bar), trans-well co-culture with HuCNS-SC (middle bar), and co-culture with HuCNS-SC plus mannose-6-phosphate (right hand bar) of (A) PPT1 enzyme activity in fibroblasts derived form CLN1 patients and (B) TPP1 enzyme activity in fibroblast derived from CLN2 patients. Mean±SEM is shown.

FIG. 2 is a graph showing the increase in whole brain PPT1 enzyme level following transplantation of different doses of HuCNS-SC. The mice used in these studies were from N6 backcross generations and spanned a range of times post-transplant (117 to 199 days). The mean PPT1 enzyme level for different dosing groups tested are shown. The characteristics of the PPT1 KO/NOD-Scid mice are described in Table 2, infra. Error bars represent the standard error of the mean and the P values above indicate the differences between groups tested by ANOVA.

FIG. 3 is a graph showing the number of autofluorescent foci in the cortex and hippocampus of three control and three transplanted (Group 1) PPT1-KO/NOD-Scid mice.

FIG. 4 is a graph showing that autofluorescence area was reduced in 5 different brain regions of NOD-Scid/PPT1−/− mice upon HuCNS-SC transplantation protocols. Four non-transplanted NOD-Scid/PPT−/− animals and three transplanted (Group 3, see Table 2, infra) are shown. Error bars represent the standard error of the mean and the P values above indicate differences between groups (correspondence for P values are indicated in the body of the graph). The 5 different brain regions are: RCtx, rostral cortex; CCtx, caudal cortex; Thal, thalamus; CA1, CA1 area of the hippocampus; and Cb, cerebellum. The average percent reduction (%) of deposit area per image field is calculated between non-transplanted controls (left bar) and transplanted PPT1-Scid recipients (right bar).

FIGS. 5A-5C are a series of photomicrographs showing that the HuCNS-SCs protect host cell neurons in PPT1-Scid mice. Human cells were transplanted in brains of PPT1-Scid mice. Brain sections were stained with MAb against NeuN. The CA area of the hippocampus showed the neuronal cell loss in a non-transplanted brain (FIG. 5A). The transplanted HuCNS-SC provided neuroprotection in the corresponding area of the transplanted mice (FIGS. 5B and 5C). The representative hippocampus pictures from Group 4 (FIG. 5B) and Group 5 (FIG. 5C) are shown. The characteristics of the cohorts of PPT1 KO/NOD-Scid mice are shown in Table 3, infra.

FIG. 5D is a graph showing the quantification of NeuN-positive staining by SIS image analysis in the CA1 area of the hippocampus above.

### DETAILED DESCRIPTION OF THE INVENTION

Lysosomal storage disorders are normally monogenic. However, for most LSDs, numerous mutations have been described in the same gene for different patients. (See Futerman and van Meer, Nature Reviews Mol. Cell Biol. 5:554-65 (2004)). The classification of many LSDs can be made either based on the characterization of the defective enzyme or protein or based on the kind of substrate that accumulates. Most LSDs exist in infantile, juvenile, and adult forms. The most severe LSDs are the infantile forms, which present with acute brain involvement. Patients suffering from infantile forms of LSDs typically die within the first years of life. In adult forms, symptoms develop more slowly and disabilities arise mainly from peripheral symptoms. Juvenile forms of LSDs fall between the infantile and adult forms.

Neurological symptoms associated with LSDs can include, for example, seizures, dementia, and brainstem dysfunction. Peripheral symptoms can include, for example, enlargement of the spleen and liver, heart and kidney injury, abnormal bone formation, muscle atrophy, and ocular disease. A summary of various LSDs is provided in Table 1.

TABLE 1

| | | |
|---|---|---|
| Disease | Defective Protein | Main Storage Materials |

Sphingolipidoses

| | | |
|---|---|---|
| Fabry | α-galactosidase A | globotriasylceramide and blood- |
| | | group-B substances |
| Farber lipogranulomatosis | Ceramidase | Ceramide |
| Gaucher | β-glucosidase | Glucosylceramide |
| | Saposin-C activator | Glucosylceramide |
| Neimann-Pick A and B | Sphingomyelinase | Sphingomyelin |
| Sphingolipid-activator deficiency | Sphingolipid activator | Glycolipids |
| GM1 gangliosidosis | β-galactosidase | GM1 ganglioside |
| GM2 gangliosidosis (Tay-Sachs) | β-Hexosaminidase A | GM2 ganglioside and related |
| | | glycolipids |
| GM2 gangliosidosis (Sandhoff) | β-Hexosaminidase A and B | GM2 ganglioside and related |
| | | glycolipids |
| GM2 gangliosidosis (GM2-activatory | GM2-activator protein | GM2 ganglioside and related |
| deficiency) | | glycolipids |

Mucopolysaccharidoses (MPS)

| | | |
|---|---|---|
| MPS I (Hurler, Scheie, Hurler/Scheie) | α-Iduronidase | Dermatan sulphate and heparan |
| | | sulphate |
| MPS II (Hunter) | Iduronate-2-sulphatase | Dermatan sulphate and heparan |
| | | sulphate |
| MPS IIIA (Sanfillipo) | Heparan N-sulphatase | Heparan sulphate |
| | (sulphamidase) | |
| MPS IIIB (Sanfillipo) | N-Acetyl-α-glucosaminidase | Heparan sulphate |
| MPS IIIC (Sanfillipo) | Acetyl-CoA: α-glucosamide N- | Heparan sulphate |
| | acetyltransferase | |
| MPS IIID (Sanfillipo) | N-Acetylgalactosamine-6- | Heparan sulphate |
| | sulphatase | |
| Morquio-A disease | N-Acetylgalactosamine-6- | Keratan sulphate, chondroitin- |
| | sulphate-sulphatase | 6-sulphate |
| Morquio-B disease | β-Galactosidase | Keratan sulphate |
| MPS VI (Maroteaux-Lamy) | N-Acetylgalactosamine-4- | Dermatan sulphate |
| | sulphatase (arylsulphatase B) | |
| MPS VII (Sly) | β-Glucuronidase | Heparan sulphate, dermatan |
| | | sulphate, chondroitin-4 and -6 |
| | | sulphates |

Oligosaccharidoses and glycoproteinosis

| | | |
|---|---|---|
| Pompe (glycogen-storage-disease type | α-Glucosidase | Glycogen |
| II) | | |

Diseases caused by defects in integral membrane proteins

| | | |
|---|---|---|
| Cystinosis | Cystinosin | Cystine |
| Danon disease | LAMP2 | Cytoplasmic debris and |
| | | glycogen |
| Infantile sialic-acid-storage disease and | Sialin | Sialic acid |
| Salla disease | | |
| Mucoplipidosis (ML) IV | Mucolipin-1 | lipids and acid |
| | | mucopolysaccharides |
| Neimann-Pick C (NPC) | NPC1 and 2 | Cholesterol and sphingolipids |

Others

| | | |
|---|---|---|
| Galactosialidosis | Cathepsin A | Sialyloligosaccharides |
| I Cell and pseudo-Hurler polydystrophy | UDP-N- | Oligosaccharides, |
| (ML II and ML III, respectively) | acetylglucosamine: lysosomal | mucopolysaccharides and lipids |
| | enzyme N-acetylglucosaminyl-1- | |
| | phosphotransferase | |
| Multiple sulphatase deficiency | Cα-formylglycine-generating | Sulphatides |
| | enzyme | |
| Neuronal ceroid lipofuscinosis (NCL)1 | CLN1 (protein | Lipidated thioesters |
| (Batten disease) | palmitoylthioesterase-1) | |
| NCL2 (Batten disease) | CLN2 (tripeptidyl amino | Subunit c of the mitochondrial |
| | peptidase-1) | ATP synthase |
| NCL3 (Batten disease) | Arginine transporter | Subunit c of the mitochondrial |
| | | ATP synthase |

### Neuronal Ceroid Lipofuscinoses

The neuronal ceroid-lipofuscinoses (NCLs) are a group of inherited, neurodegenerative, lysosomal-storage disorders characterized by intracellular accumulation of fluorescent ceroid lipofuscin storage material, in neurons and other cells. NCLs are characterized by progressive cognitive and motor deterioration, blindness, seizures, and early death. Thus far, no curative treatment is available.

The NCL disorders are classified as lysosomal storage diseases. The classification of NCL disorders into various disease subtypes has traditionally relied on phenotypical manifestations such as age of onset, order of appearance of clinical features, and morphology of lysosomal material under light and electron microscopy. This classification describes four subtypes: infantile neuronal ceroid lipofuscinosis (“INCL”), late infantile (“LINCL”), juvenile (“JNCL”), and adult (“ANCL”). Worldwide, the most common forms of NCL are INCL and LINCL. The NCL group of disorders is commonly referred to as Batten disease. Infantile and late-infantile neuronal ceroid lipofuscinoses are the most severe forms of Batten disease.

Infantile NCL (INCL), also known as Haltia-Santavuori disease or CLN1, was first described by Santavuori and co-workers in 1973. (See Santavuori et al., J. Neurol Sci 18:257-67 (1973)). The first symptoms manifest around the age of 1 year as muscular hypotonia, regression in motor and cognitive function, and progressive microencephaly. Irritability and sleep disorders are also common signs in the early phases. Visual failure is noticed between the ages of 12 and 22 months and rapidly leads to blindness. Epileptic seizures and myoclonic jerks are prominent. The condition of subjects suffering from INCL rapidly deteriorates, and, by the age of 3 years, all cognitive and motor skills are lost. Death usually occurs between 8 and 11 years of age. The highest incidence of INCL worldwide occurs in Finland.

Classic late infantile NCL (LINCL), also known as Jansky-Bielchowsky disease or CLN2, was originally described in 1908 by Jansky and in 1913 by Bielchowsky. However, Jansky and Bielchowsky were unable to separate this type of NCL from the forms with later onset. (See Wisniewski et al, Neuronal ceroid lipofuscinoses: Classification and diagnosis. In: Batten Disease: Diagnosis, Treatment and Research. Wisniewski et al. (Eds.), San Diego: Academic Press (2001)). With LINCL, the onset of the disease appears between the ages of 2 and 4 years. The first sign of LINCL is usually epilepsy. Sometimes, delayed speech may precede the onset of epilepsy. Additional symptoms include dementia, ataxia, and myoclonic jerks. Visual failure leads to blindness usually by 5 or 6 years of age. Death usually occurs between 6 and 15 years of age. Although LINCL is rare in Finland, it is one of the most common NCL types in the United States and Canada.

Children diagnosed with any form of Batten disease suffer seizures and progressive loss of motor skills, sight, and mental capacity, eventually becoming blind, bedridden and unable to communicate. Today, Batten disease is always fatal.

### Therapeutic Effects in Neuronal Ceroid Lipofuscinoses

During development of the central nervous system (“CNS”), multipotent precursor cells (also known as neural stem cells) proliferate and give rise to transiently dividing progenitor cells that eventually differentiate into the cell types that compose the adult brain. Neural stem cells are classically defined as having the ability to self-renew (i.e., form more stem cells), to proliferate, and to differentiate into multiple different phenotypic lineages, including neurons, astrocytes and oligodendrocytes.

The non-stem cell progeny of a neural stem cell are typically referred to as “progenitor” cells, which are capable of giving rise to various cell types within one or more lineages. Thus, the term “neural progenitor cell” refers to an undifferentiated cell derived from a neural stem cell, and is not itself a stem cell. Some progenitor cells can produce progeny that are capable of differentiating into more than one cell type. A distinguishing feature of a progenitor cell is that, unlike a stem cell, it does not exhibit self maintenance, and typically is thought to be committed to a particular path of differentiation and will, under appropriate conditions, eventually differentiate into glia or neurons.

The term “precursor cells” refers to the progeny of neural stem cells, and thus includes both progenitor cells and daughter neural stem cells.

Neural stem cells have been isolated from several mammalian species, including mice, rats, pigs and humans. See, e.g., WO 93/01275; WO 94/09119; WO 94/10292; WO 94/16718; U.S. Pat. No. 5,968,829; and Cattaneo et al., Mol. Brain Res., 42, pp. 161-66 (1996), all herein incorporated by reference.

A population of cells exists within the adult CNS, which exhibit stem cell properties, in their ability to self-renew and to produce the differentiated mature cell phenotypes of the adult CNS. These stem cells are found throughout the CNS and particularly in the subventricular regions, and dentate gyrus of the hippocampus. Growth factor-responsive stem cells can be isolated from many regions of the neuraxis and at different stages of development, of murine, rodent and human CNS tissue. These cells vary in their response to growth factors such as EGF, basic FGF (bFGF, FGF-2) and transforming growth factor alpha (TGFα), and can be maintained and expanded in culture in an undifferentiated state for long periods of time. The identification, culture, growth, and use of mammalian, including human, neural stem cell cultures, either as suspension cultures or as adherent cultures, is disclosed in Weiss et al., U.S. Pat. No. 5,750,376 and Weiss et al., U.S. Pat. No. 5,851,832, both incorporated herein by reference. Similarly, Johe, U.S. Pat. No. 5,753,506, incorporated herein by reference, refers to adherent CNS neural stem cell cultures. When cultured in suspension, CNS neural stem cell cultures typically form neurospheres.

The cells of a single neurosphere are clonal in nature because they are the progeny of a single neural stem cell. In the continued presence of a proliferation-inducing growth factor such as EGF or the like, precursor cells within the neurosphere continue to divide resulting in an increase in the size of the neurosphere and the number of undifferentiated neural cells. Neurospheres are not immunoreactive for neurofilament (NF; a marker for neurons), neuron-specific enolase (NSE; a marker for neurons) or myelin basic protein (MBP; a marker for oligodendrocytes). However, cells within the neurosphere are immunoreactive for nestin, an intermediate filament protein found in many types of undifferentiated CNS cells. (See Lehndahl et al., 60 Cell 585-595 (1990), incorporated herein by reference). Antibodies are available to identify nestin, including the rat antibody referred to as Rat401. If the neurospheres are cultured in conditions that allow differentiation, the progenitor cells differentiate to neurons and glia. The mature phenotypes associated with the differentiated cell types that may be derived from the neural stem cell progeny are predominantly negative for the nestin phenotype.

Human central nervous system stem cell-derived neurospheres (“HuCNS-SC™”) (StemCells, Inc., Palo Alto, Calif.) are a somatic cell therapy product comprised of a homogeneous aseptic suspension of neural progenitor cells capable of migrating from the implantation site and differentiating into mature cell types of the brain. HuCNS-SCs are under development as a cell therapy for the treatment of signs and symptoms associated with neuronal ceroid lipofuscinosis (NCL) in subjects with deficiencies in the lysosomal enzymes CLN1-encoding palmitoyl protein thioesterase 1 (PPT1) and CLN2-encoding tripeptidyl peptidase I (TPP-I).

The neuronal ceroid lipofuscinoses (NCLs) include several types of lysosomal storage disorders that are distinguished from each other by the onset of clinical symptoms determined by the inherited genetic mutations in various genes. The consequence of these mutations is the accumulation of lipofuscin-like fluorescent inclusions in various cell types, which eventually leads to cell degeneration. (See Goebel, J. Child Neurol 10:424-37 (1995)). The infantile NCL carries mutations in the CLN1 gene (see Vesa et al., Nature 376:584-87 (1995); Schriner et al., Genomics 34:317-22 (1996)), which codes for palmitoyl-protein thioesterase 1 (PPT1). The late infantile NCL carries mutations in the CLN2 gene (see Sharp et al., Hum Mol Genet 6:591-95 (1997); Sleat et al., Science 277:1802-05 (1997)), which codes for tripeptidyl peptidase I (TPP-1). PPT1 enzyme hydrolyses the thioester linkage between the palmitoyl group and the sulphur atoms of cysteine amino acid residues, while TPP-I has been proposed to be a member of the sedolisin family of serine-carboxyl peptidases. (See WIodawer et al., BMC Struc Biol 3:8-10 (2003); Tomkinson, TIBS 24:355-59 (1999)). Humans having mutations in the CLN1 gene have been shown to develop INCL disease symptoms when functional PPT1 enzyme levels are approximately below 3% of normal enzymatic levels.

The two enzymes, PPT1 and TPP-I, are classified as classical soluble lysosomal hydrolases that are routed from the rough endoplasmic reticulum (RER) to the lysosomes through the mannose 6-phosphate receptor protein-sorting pathway. The newly synthesized hydrolases are secreted secondarily because a certain percentage escape recognition by the mannose 6-phosphate receptor in the RER and end up in secretion vesicles. The extracellular enzymes specifically bind to cell surface mannose 6-phosphate receptors, and the complex is internalized and directed to the lysosomes. The acidic pH of the lysosomes causes the proteins to dissociate from the receptor, and the 6-phosphate group on mannose is, in turn, removed by lysosomal phosphatases to ensure that the internalized proteins remain and accumulate in the lysosomes and allows the receptor to recycle back to the ER.

TPP-I is synthesized as a precursor protein (see Golabek et al., J Biol Chem 278:713545 (2003)) and, therefore, is inactive until autocatalytically cleaved and converted to the active form in the lysosomes. It has previously been demonstrated that over-expressed, secreted, recombinant PPT1 and TPP-I enzymes can be internalized by mammalian cells. (See Verkruyse and Hoffman, J Biol Chem 271:15831-36 (1996); Bellizzi et al., Proc Natl Acad Sci USA 97:4573-78 (2000); Lehtovirta M. et al., Hum Molec Genet 10:69-75 (2001); Lin and Lobel, Biochem J. 357:49-55 (2001)). Receptor-dependent endocytic uptake is shown to be mediated specifically through the mannose 6-phosphate receptor present on the cell surface and mannose 6-phosphate inhibits both PPT1 and TPP-I internalization.

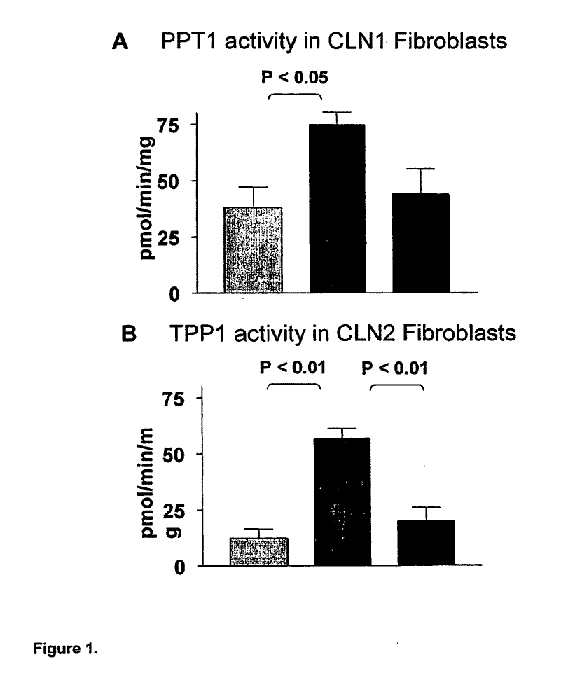

HuCNS-SC have been shown to constitutively synthesize and secrete both PPT1 and TPP-I enzymes under standard culture conditions, as evidenced by detection of enzyme activity in cell lysates and culture media. (See FIG. 1). The PPT1 and TPP-I enzymes accumulate in the lysosomal compartment of human cells and a portion of enzyme that is secreted can be endocytosed into fibroblasts of patients harboring either CLN1 or CLN2 gene mutations, respectively. Competitive inhibition of the mannose-6-phosphate receptor, the natural receptor for these enzymes, blocks receptor-mediated endocytosis in this experimental system.

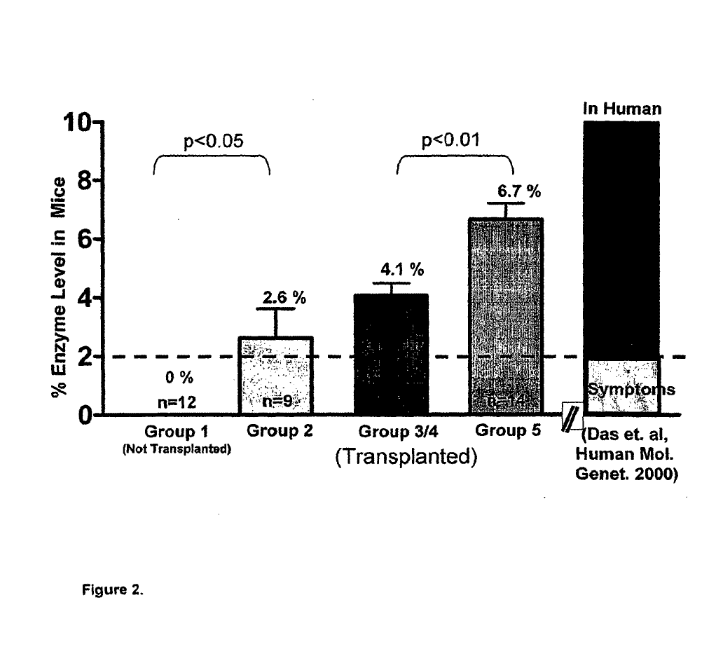

In vivo, neurospheres establish long-term engraftment in the developing brains of neonatal NOD.CB17-PrkdcScid/J (NOD-Scid) strain of mouse. These cells migrate into regions distal from the site of implantation and differentiate into GABAergic and tyrosine hydroxylase-immunoreactive neurons, astrocytes and oligodendrocytes. In an animal model of genetic PPT1 deficiency (CLN1 gene knock-out backcrossed to the NOD-Scid genetic background (PPT1-Scid)), transplantation of HuCNS-SC resulted in substantial engraftment and enzyme secretion. (See FIG. 2), HuCNS-SC transplanted into PPT1 K/O Scid mice migrate, differentiate and produce enzyme in this well-described neural degeneration model.

### Therapies for Lysosomal Storage Disorders

Enzyme-Replacement Therapy

Currently, enzyme-replacement therapies are used to treat lysosomal storage disorders. Such therapies utilize the ability of cells to internalize lysosomal proteins through the cell surface mannose 6-phosphate receptor pathway. (See Germain, Expert Opin. Investig. Drugs 11:1467-76 (2002); Bengtsson et al., Lancet 361:352 (2003)). These therapies have been effective in treating symptoms associated with the peripheral system. However, symptoms associated with the central nervous system (CNS) have proven to be difficult to alleviate due to the impermeability of the blood-brain barrier to the enzymes used in enzyme-replacement therapy.

HuCNS-SC Therapy

The failure of enzyme-replacement therapy to treat LSD symptoms associated with the CNS can be overcome by delivering the enzyme directly to the CNS of patients by transplanting HuCNS-SCs into the CNS of patients. HuCNS-SCs naturally produce and secrete soluble lysosomal enzymes, including TPP-I and PPT1. (See FIG. 1). Thus, upon dissemination and engraftment of HuCNS-SCs in the CNS, the cells would serve as a continuous and permanent source of soluble lysosomal enzymes for the CNS.

HuCNS-SC have been shown to produce both PPT1 and TPP-I enzymes. (See FIG. 1). Moreover, in preclinical models of PPT1 deficiency, the corresponding enzyme activity increases with time after transplantation. Thus, placement of HuCNS-SC in appropriate places in the brains of patients suffering from INCL Or LINCL can be used to replace these missing enzymes.

Other examples of soluble lysosomal enzymes that lead to lysosomal storage disorders when inactivated due to genetically inherited mutations in their genes include, for example, β-glucocerebrosidase, α-L-iduronidase, and sulfamidase. Specifically, mutations in β-Glucocerebrosidase lead to Gaucher disease due to the accumulation of undegraded glucosylceramide in the lysosomes. Likewise, defects in α-L-iduronidase enzyme cause Hurler (MPS I) disease where dermatan sulfate and heparan sulfate material build up in the lysosomes. Finally, lack of sulfamidase enzyme results in Sanfilippo (MPS IIIA) disease, where heparan sulfate accumulates in the lysosomes. Other examples include Tay Sachs, Sandhoff and Hunter's diseases. Additional examples are also detailed in Table 1, supra.

It is well recognized in the art that transplantation of tissue into the CNS offers the potential for treatment of neurodegenerative disorders and CNS damage due to injury. (See Lindvall, (1991) TINS vol. 14(8): 376-383). Moreover, as described herein, transplantation of HuCNS-SC offers the potential for the treatment of lysosomal storage disorders.

Transplantation of new cells into the CNS has the potential to repair damaged circuitries and to provide deficient, defect, or missing biologically active molecules, thereby restoring function. However, the absence of suitable cells for transplantation purposes has prevented the full potential of this procedure from being met. “Suitable” cells are cells that meet the following criteria: 1) can be obtained in large numbers; 2) can be proliferated in vitro to allow insertion of genetic material, if necessary; 3) capable of surviving indefinitely but stop growing after transplantation to the brain; 4) are non-immunogenic, preferably obtained from a patient's own tissue; 5) are able to form normal neural connections and respond to neural physiological signals. (See Bjorklund (1991) TINS Vol. 14(8): 319-322). The progeny of multipotent neural stem cells obtainable from embryonic or adult CNS tissue, which are able to divide indefinitely when maintained in vitro meet all of the desirable requirements of cells suitable for neural transplantation purposes and are a particularly suitable cell line as the cells have not been immortalized and are not of tumorigenic origin.

HuCNS-SC can be administered to any animal with abnormal neurological or neurodegenerative symptoms obtained in any manner. Moreover, HuCNS-SC can also be administered to patients suffering from a lysosomal storage disorder.

In some instances, it may be possible to prepare HuCNS-SC from the recipients own nervous system (e.g., in the case of tumor removal biopsies etc.). In such instances, the neural stem cell progeny may be generated from dissociated tissue and proliferated in vitro using any suitable method known to those of ordinary skill in the art. Upon suitable expansion of cell numbers, the HuCNS-SC cells may be harvested, genetically modified if necessary, and readied for direct injection into the recipient's CNS.

HuCNS-SC, when administered to the particular neural region, preferably form a neural graft, wherein the neuronal cells form normal neuronal or synaptic connections with neighboring neurons, and maintain contact with transplanted or existing glial cells which may form myelin sheaths around the neutons' axons, and provide a trophic influence for the neurons.

Survival of the graft in the living host can be examined using various non-invasive scans such as computerized axial tomography (CAT scan or CT scan), nuclear magnetic resonance or magnetic resonance imaging (NMR or MRI) or more preferably positron emission tomography (PET) scans. Post-mortem examination of graft survival can be done by removing the neural tissue, and examining the affected region macroscopically, or more preferably using microscopy. Cells can be stained with any stains visible under light or electron microscopic conditions, more particularly with stains which are specific for neurons and glia. Particularly useful are monoclonal antibodies which distinguish and/or identify donor from host cells, specifically differences in H-2 or HLA histocompatibility antigens. Most preferable are antibodies which identify any neurotransmitters, particularly those directed to GABA, TH, ChAT, and substance P, and to enzymes involved in the synthesis of neurotransmitters, in particular, GAD. Transplanted cells can also be identified by prior incorporation of tracer dyes such as rhodamine- or fluorescein-labelled microspheres, fast blue, bisbenzamide or retrovirally introduced histochemical markers such as the lac Z gene which produces beta galactosidase.

Functional integration of the graft into the host's neural tissue can be assessed by examining the effectiveness of grafts on restoring various functions, including but not limited to tests for lysosomal function.

For transplants into human patients, those skilled in the art will recognize that any suitable method for the transplantation, administration, injection, and/or implantation of HuCNS-SC can be employed in patients. (See, e.g., U.S. Pat. No. 6,497,872, incorporated herein by reference).

A range of between about 3×10⁶ to about 1×10¹⁰HuCNS-SC cells, for example between about 5×10⁸ to about 2×10⁹ cells or about 1×10⁸ to about 5×10⁹ cells, can be administered to a mammal suffering from a lysosomal storage disorder. Specifically, a low dose of 5×10⁸ cells or a high dose of 1×10⁹ cells can be transplanted or implanted or injected or administered to the mammal. Those skilled in the art will recognize that transplantation can be accomplished using any neurotransplantation protocols known to those skilled in the art. (See, e.g., U.S. Pat. No. 6,497,872, incorporated herein by reference).

HuCNS-SC are administered in 8 specific regions of the patient's CNS, including the lateral ventricles, and the frontal, parietal, and parietal/occipital regions of the cortex in each hemisphere of the brain. HuCNS-SC are implanted into the brain through a surgical procedure consisting of six bilateral sub-cortical injections and two bilateral intra-ventricular injections. The procedure is conducted in the operating room under general anesthesia by a pediatric neurosurgeon. Three trephine holes are made over each cerebral hemisphere. The trephinations are centered over the medial aspects of the frontal and parietal lobes. Patients will receive either 5×10⁷ cells/cortical trephine and 1×10⁸ cells/ventricle trephine (for a total dose of 5×10⁸ HuCNS-SC per subject) or 1×10⁸ cells/cortical trephine and 2×10⁸ cells/ventricle trephine (for a total dose of 1×10⁹ HuCNS-SC per subject).

The invention will be further described in the following examples, which do not limit the scope of the invention described in the claims.

### EXAMPLES

### Example 1

### Transplantation of HuCNS-SC in CLN1 and CLN2 Patients

Human CNS stem cells (HuCNS-SC) are a cell therapy product comprised of an injectable suspension of human neural stem/progenitor cells. HuCNS-SC are transplanted in the CLN1 and CLN2 patients in part to determine if the transplanted cells secrete the missing lysosomal enzymes into the brains of affected individuals. HuCNS-SC have been shown to produce both PPT1 and TPP-I enzymes, thereby providing a scientific justification for enzyme replacement and cellular rescue in this indication. In preclinical models of PPT1 deficiency, the corresponding enzyme activity increases with time after transplantation. Thus, the safety of HuCNS-SC in the treatment of infantile and late-infantile neuronal ceroid lipofuscinosis (NCL), the most severe forms of a group of disorders commonly referred to as Batten disease, can be investigated.

HuCNS-SC Transplantation

A range of between about 3×10⁶ to about 1×10¹⁰ HuCNS-SC cells, for example between about 5×10⁸ to about 2×10⁹ cells or about 1×10⁸ to about 5×10⁹ cells, can be administered to a mammal suffering from a lysosomal storage disorder. For example, HuCNS-SC are surgically administered by subcortical and intraventricular injection. Two doses of cells are administered: a low dose of 5×10⁸ cells injected at a concentration of 5×10⁷ cells/ml and a high dose of 1×10⁹ cells injected at a concentration of 1×10⁸ cells/ml.

Preoperative MRI is used to select subcortical target sites in the anterior frontal, anterolateral frontal, and parietal lobes where the cortical mantle (brain surface to ventricular surface) is at least 20-30 mm thick. Target sites are selected so as to avoid eloquent cortex and other critical brain structures. Cortical thickness is measured directly off the MRI scan images. Four burr holes are placed on each side of the skull, three for access to the selected subcortical sites and one for access to the lateral ventricle. A stereotactic navigation instrument such as the StealthStation® (Medtronic, 510K No. K001153) may be used in addition to anatomic landmarks to aid in the anatomic localization of the burr holes corresponding to the targeted subcortical injection sites. The stereotactic navigation instrument will only be used for planning purposes and as an aide in locating anatomic structures; it will not be used for injection of HuCNS-SC.

HuCNS-SC are injected subcortically to a depth of approximately 20 mm below the cortical surface. One ml of HuCNS-SC suspension is injected manually over 3-5 minutes. The rate of injection is hand-modulated based on the ability of the brain to absorb the volume without visible reflux back along the needle track. The needle is left in place for 2-3 minutes after each injection and then withdrawn slowly.

For the ventricular injections, the frontal horn of the lateral ventricle is cannulated. The selected catheter should be a well established neurosurgical instrument that is used for atraumatic access to the ventricle and for injection of antibiotics, chemotherapeutics or dye into the ventricle. Approximately 5 ml of the patients CSF are withdrawn through the catheter and set aside to be used to flush the catheter after injection. Two ml of HuCNS-SC suspension is injected manually over 2-3 minutes. The catheter is flushed with 2 ml of the patients CSF over 2-3 minutes and then slowly removed.

At the conclusion of the procedure, each burr hole is closed by placing Surgifoam absorbable gelatin sponge (Ethicon, PMA No. P990004) in the burr hole above the dura, and the galea closed with Vicryl sutures (Ethicon) and the skin closed with Monocry sutures (Ethicon). Patients are monitored in the pediatric intensive care unit at least overnight after surgery.

Immunosuppression

HuCNS-SC Cell Therapy is an allogeneic transplant. The cells are implanted into subjects without donor and recipient tissue-type matching. Thus, immunosuppression may be necessary to prevent rejection of the transplanted HuCNS-SCs.

For example, combination immunosuppression therapy using corticosteroids (10 mg/kg/day) and Prograf® (0.3 mg/kg/day) may be employed for up to 1 year post-transplant. Specifically, Prograf® can be initiated prior to the transplant and maintained up to one year post-transplant (dosage is reduced to 0.1 mg/kg/day 30 days following transplant). Prograf® administration can be monitored for adverse experiences at specific intervals post-transplant to assess tolerability. Toxicokinetic assessment of Prograf® blood levels will permit customized dosing for each subject. In addition, corticosteroids can be administered immediately prior to surgery for up to 5 days post-operatively and then tapered to discontinuation.

### Example 2

### Pharmacology Study of Intracranial PPT1 Enzyme Activity

As shown in FIG. 2, many studies have been conducted using different doses and regimens of HuCNS-SC transplantation in PPT1-KO/NOD-Scid mice. As used herein, the terms “PPT1-KO/NOD-Scid mice” and “CLN1 mice” are used interchangeably to refer to the PPT1−/− knockout mice. FIG. 2 includes data examining the effect of higher dose levels and multiple transplants on PPT1-KO/NOD-Scid mice.

In the PPT1-KO/NOD-scid mouse model, endogenous PPT1 enzyme is below the level of specific detection. Studies transplanting doses of 3-8×10⁵ HuCNS-SC into neonatal PPT1-KO/NOD-Scid mice cells (Group 2) yielded an average of 2.6% of the whole brain PPT1 enzyme level. Because of the limited brain mass of the neonatal mouse, sequential transplant schemes were developed to deliver higher doses of HuCNS-SC into the brain of these animals. Group 3/4 transplants (neonatal plus postnatal or juvenile) were used to administer 1.5-2.0×10⁶ cells and Group 5 transplants (neonatal, postnatal and juvenile) were used to administer 2.8×10⁶ cells. Some animals in these experiments had HuCNS-SC targeted to the cerebellum and/or hippocampus. PPT1 enzyme level in the double transplants averaged 4.1% and in the triple transplants, enzyme levels averaged 6.7% (see FIG. 2 and Table 2 for the characteristics of the different cohorts of PPT1 KO/NOD-Scid mice). In the experiments described herein, Group 1 included the non-transplanted control PPT1-Scid mice as a negative control (“not-transplanted group”); Group 2 included mice transplanted a single time as neonates (P0-P1) (“single transplant group”); Group 3 included mice transplanted once as neonates (P0-P1) and again postnatally (P7-P8), (“double transplants-NP group”); Group 4 included mice transplanted as neonates, and again at juvenile-young adult as described (“double transplants-NJ group”); and Group 5 included mice transplanted as neonates, postnatally (P7-P8) and at juvenile-early adult (“triple transplants group”).

TABLE 2
Characteristics of the cohorts of PPT1-Scid mice

| Cohort | Transplantation | Cell Dose |
|---|---|---|
| Group 1 | Non-transplanted PPT1-Scid | — |
| Group 2 | Single transplant | 0.3-0.8 × 10⁶ |
| Group 3 | Double transplants-NP | 1.6 × 10⁶ |
| Group 4 | Double transplants-NJ | 1.5-2.0 × 10⁶ |
| Group 5 | Triple transplants | 2.8 × 10⁶ |

### Example 3

### Pharmacology Study of Reduction of Autofluorescent Lipofuscin Accumulation

A hallmark of the PPT1−/− mouse pathology is the accumulation of lipofuscin deposits in neutrons and other cells throughout the brain. The mutant mice have progressive neurodegeneration, which can be characterized as pathological changes and neuronal cell loss in the cortex and the hippocampus. (See Bible et al., Neurobiol Dis 16(2):346-59 (2004)).

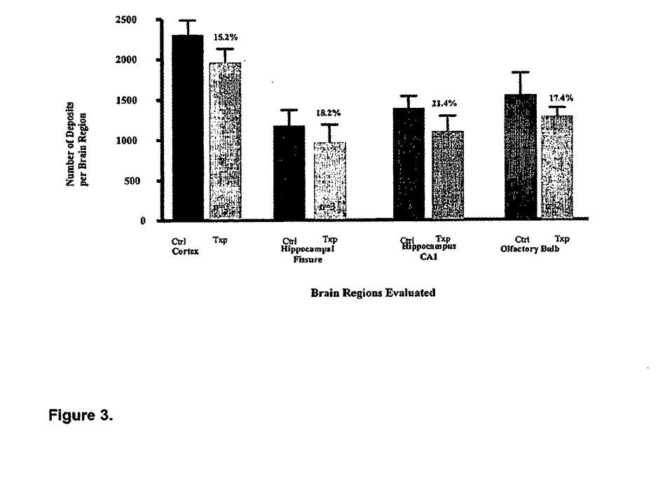

A pilot study quantified the amount of autofluorescence in mice that were treated with 400,000 cells (n=3) injected into the lateral ventricle and cerebellum as a neonate and sacrificed 160-167 days post transplant (one animal received a second transplant of an additional 100,000 cells into the hippocampus one day before sacrifice). Compared to non-transplanted control brains (n=3), animals treated with HuCNS-SC showed a 15% reduction in storage material within the cortex, and a 21% decrease in the hippocampus CA1 region. The numbers of mice involved in this pilot study were small and the results were not statistically significant. (See FIG. 3).

Dose effects on a biologic marker of the disease were conducted by analyzing the amount of autofluorescent storage material that accumulates in the brain of HuCNS-SC transplanted PPT1-KO/NOD-Scid mice compared to non-transplanted controls. Autofluorescent lipofuscin load was measured in non-transplanted PPT1-KO/NOD-Scid mice (n=4) and mice transplanted with 1.5-1.8×10⁶ HuCNS-SC cells (n=3, Group 4). Lipofuscin accumulation was quantified as the average area (μm²) that was autofluorescent per image field in the rostal cortex, caudal cortex, thalamus, CA1 region of the hippocampus and cerebellum. The average percent reduction of autofluorescent in each brain region was calculated between untransplanted controls, and transplanted PPT1-KO/NOD-Scid recipients.

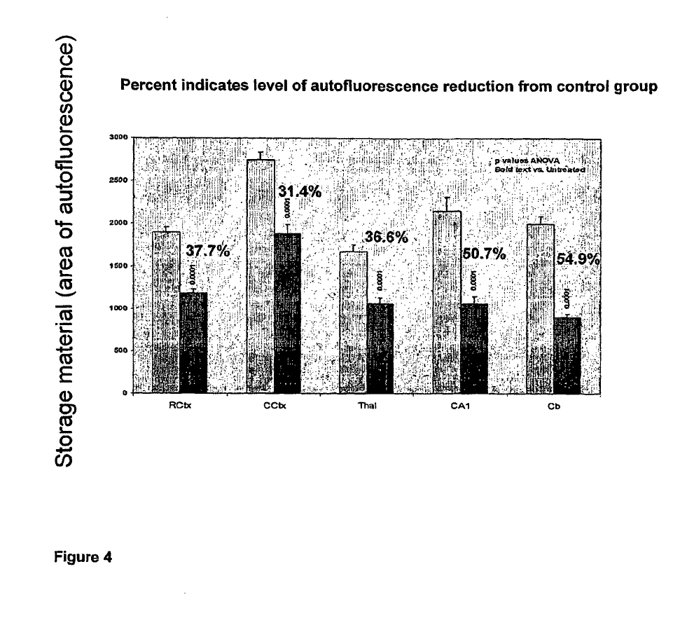

Significant reduction in autofluorescent deposits was obtained in transplanted mice in all areas of the brain measured (P=0.0001; see FIG. 4). The percentage of reduction in autofluorescent deposits as compared to controls ranged from 31% in the caudal cortex to 54% in the cerebellum.

### Example 4

### Characterization of Neuroprotection by Transplantation of HuCNS-SC

PPT1 KO/NOD-Scid (hereinafter “PPT1−/−” or “PPT1-Scid” or “CLN1”) mice were backcrossed onto the NOD-Scid background for six generations (N6). To overcome brain volume as a limitation of cell dosing, PPT1-Scid mice were transplanted multiple times over the first several weeks of life, from birth to juvenile (early adult). Brains of mice transplanted with different doses of cells were chosen for analysis of neuroprotection as represented in Table 3. Group 1 included the non-transplanted control PPT1-Scid mice as a negative control (n=9) (“not-transplanted group”); Group 2 included mice transplanted a single time as neonates (P0-P1) (n=3) (“single transplant group”); Group 3 included mice transplanted once as neonates (P0-P1) and again postnatally (P7-P8), (n=5) (“double transplants-NP group”); Group 4 included mice transplanted as neonates, and again at juvenile-young adult as described (n=6) (“double transplants-NJ group”); Group 5 included mice transplanted as neonates, postnatally (P7-P8) and at juvenile-early adult (n=6) (“triple transplants group”); and Group 6 included a non-transplanted NOD-Scids, as a control for normal NeuN staining (n=2) (NOD-Scid (“PPT1+/+group”)).

TABLE 3
Characteristics of the three cohorts of PPT1-Scid
mice for quantification of NeuN staining

| | | | Age at sacrifice |
|---|---|---|---|
| Cohort | Transplantation | Cell Dose | (days) |
| Group 1 n = 9 | Non-transplanted | — | 166-171 |
| | PPT1-Scid | | |
| Group 2 n = 3 | Single transplant | 0.3-0.8 × 10⁶ | 168-176 |
| Group 3 n = 5 | Double transplants-NP | 1.6 × 10⁶ | 174-177 |
| Group 4 n = 6 | Double transplants-NJ | 1.5-2.0 × 10⁶ | 167-180 |
| Group 5 n = 6 | Triple transplants | 2.8 × 10⁶ | 165-188 |
| Group 6 n = 2 | NOD-Scid control | — | 294 |

At sacrifice, mice were anesthetized and transcardially perfused with phosphate buffered saline (PBS). Brain hemispheres were fixed for 24 h in 4% paraformaldehyde and cryoprotected for 48 h in 30% sucrose solution. The fixed brain hemisphere was sectioned at 40 pm thickness on a freezing sliding microtome. Sections were collected into 96 well plates (1 section per well). Every sixth 40 μm sagittal section was stained with MAb against NeuN (1:5000, Chemicon International), followed by incubation with a biotinylated goat anti-mouse IgG and the components of the VECTASTAIN ELITE ABC KIT (Vector, Burlingame). The antibody-immunoperoxidase complex was revealed using the NovaRED substrate (Vector, Burlingame). Brain sections were mounted on glass slides and counter stained with methyl green.

Image Acquisition and Analysis

All histological sections utilized in this study were imaged using an Olympus BX61 fully automated research microscope equipped with the Olympus DP70 12-bit cooled digital color camera.

CNS substructures in sagittal sections of the host brain were defined prospectively as the region of interest (“ROI”), and used for image capture and quantitative analysis. The ROI which encompasses the CA1 field of the hippocampus is referred to as HC-CA1. The CA2 and CA3 fields of hippocampus were combined (CA2/3) and referred to as HC-CA2/3.

The ROI which encompasses the cortex was delineated according to conventional histological landmarks from an anterior boundary at the ventral orbital cortex to the posterior boundary at the retrosplenial cortex and ventrally at its boundary with the corpus callosum. The ROI which encompasses the cortex may also be referred to as CRTX.

Quantitative image analysis was performed using the Soft Imaging System (SIS) GmbH Biological Suite with Scopeview software. For quantification, a series of sagittal brain sections in a given mouse was examined from medial to lateral orientation. The sections with appropriate architecture within the defined landscape were selected for image analysis. Between 5 and 9 sections per brain were used to quantify NeuN staining. The total stained areas in a given ROI per section were quantified by SIS image analysis for all sections. The mean of total stained area in the ROI for different groups were calculated with standard error.

Statistical Analysis

All data points were analyzed by one-way ANOVA followed by the Bonferroni post-test. Separate statistical analyses were performed for each region of interest, HC-CA1, HC-CA2/3 and CRTX. Statistical significance of differences between control and treated (transplanted) groups was accepted at P<0.05.

### Results

Characterization of NeuN Positive Cells in PPT1-Scid Mouse Brains

In this study, it was demonstrated that transplanted HuCNS-SCs can protect host neurons from degeneration in brains of CLN1 mice. The experiments focused on the CA1 and CA2/3 regions of the hippocampus and the cortex of transplanted PPT-1-Scid (CLN1) mice, as these areas are severely affected in human CLN1 patients.

CLN1 mice, backcrossed into NOD-Scid background, undergo progressive neurodegeneration over their life-span and prematurely die at age approximately 24 weeks (168 days). As early as 6 weeks of age, CLN1 mice begin to accumulate high levels of autofluorescent compounds known as lipofuscin. The autofluorescent material accumulates in neurons throughout the life time of CLN1 mice and is associated with neuronal cell death especially in the cortex and the CA regions of the hippocampus (Gupta et al., Proc Natl Acad Sci USA 98(24):13566-71 (2001); Bible et al., Neurobiol Dis 16(2):346-59 (2004)). The brains of diseased animals are greatly atrophied at the end stage of life.

Mice that were examined ranged in age from 165-188 days. Non-transplanted PPT1-Scid control and transplanted cohorts overlap for the age at which they were examined.

Several markers of mature neurons, such as calbindin, calretinin and NeuN were screened. NeuN expression was widely distributed in the cortex and hippocampus and staining was localized to the cell bodies of the neurons. NeuN (neural nuclear antigen) is a DNA binding protein that is expressed in the nuclei and perinuclear cytoplasm of most post mitotic neurons. NeuN is not expressed in Purkinje cells, mitral cells and photoreceptors in mice. Commercially available anti NeuN antibodies are immunoreactive with both rodent and human forms of NeuN. However, these commercial antibodies react more strongly with mouse neurons and, thus, can be titrated to preferentially stain only mouse neurons.

Double-labeling experiments were conducted with NeuN and human specific mAb SC121. SC121 recognizes a cytoplasmic antigen and give a variety of morphological characteristics of engrafted human cells in rodent and non-human primate brains. (See Kelly, Proc. Natl Acad Sci USA 101:11839-44 (2004); Cummings, Proc Natl Acad Sci USA 102:14069-74 (2005)). The cortex and hippocampus of transplanted PPT1-Scid mice were stained with NeuN and SC 121 antibodies and analyzed by confocal microscopy. The image stacks were inspected in the z-dimension using the orthogonal view tool or 3-D rendering tool available in the Volocity.

NeuN staining is primarily restricted to the cell bodies of mouse neurons. None of the SC121 positive human cells that are engrafted in the cortex or hippocampus of transplanted PPT1-Scid mice were NeuN positive. However, of the human cells that remained in the injection core, NeuN positive cells were occasionally detected. The cell density in the injection core was too high and individual NeuN positive cells could not be distinguished to determine whether they were of mouse or human origin. Therefore, the injection cores were excluded from the defined analytical region of interest. In the context of this study, it is believed that NeuN positive cells are mouse host cells.

Brain sections were treated with anti NeuN antibody and detected with immunoperoxidase staining method. FIG. 4 shows representative NeuN staining from (A) non-transplanted (NT, Group 1), (B) double transplanted at neonatal and juvenile (DT-NJ, Group 4), and (C) triple transplanted at neonatal, postnatal and juvenile (TT, Group 5). Qualitatively, the NeuN staining of the hippocampus of non-transplanted mice (A) reveals that the CA regions, especially CA1, bad greatly reduced staining indicating neuronal cell loss. More NeuN immunoreactivity is seen in PPT1-Scid animals receiving either double or triple transplants (B & C), thereby strongly suggesting neuroprotection of host cells by the transplanted hCNS-SC. Based on these observations, the area of NeuN positive staining was quantified as a measure of neuroprotection.

Quantification of NeuN Positive Cells

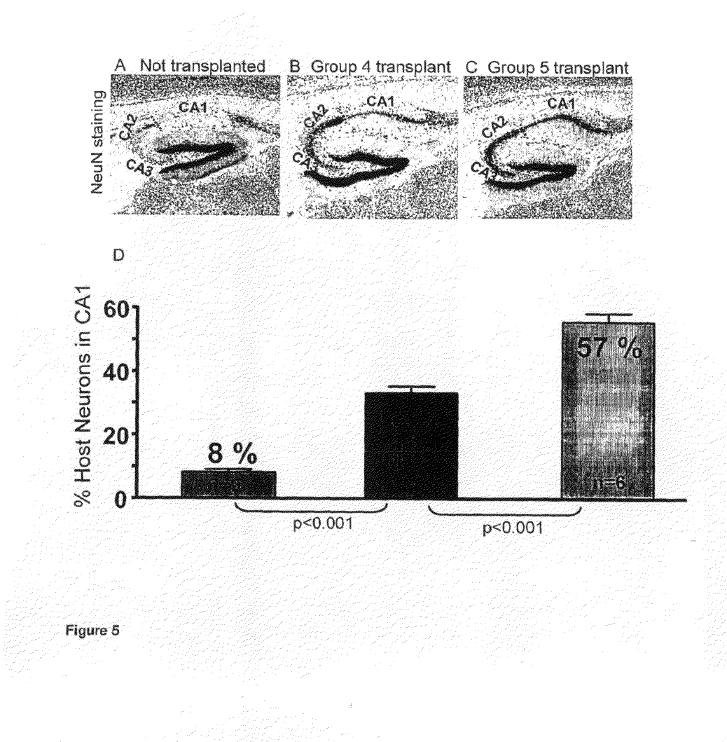

The CA1 region of the CLN1 mouse hippocampus consists of distinct layers of pyramidal neurons which makes it ideal for quantitative image analysis. The sagittal sections of mouse brains were stained with anti-NeuN antibody and the total stained areas in the CA1 region per section were quantified by SIS image analysis for all sections. The mean of total stained area in the CA1 region of the hippocampus for different transplanted groups were calculated with standard error. (See FIG. 5D).

In non-transplanted PPT1-Scid controls, only 8% of host neurons survive at the time point examined, as compared to NOD-Scid animals. In all transplanted groups, there is a significantly high level of NeuN positive neurons, as compared to PPT1-Scid non-transplanted controls. As much as 57% of the area of NeuN positive cells was present in mice which received the highest cell dose (Group 5). The NeuN stained area was increased with increasing number of transplanted HuCNS-SCs. The group with double transplants-NP (neonatal and postnatal) bad an unexpectedly lower amount of the area of NeuN positive cells. This group should have a NeuN positive level comparable to the level of the single transplant group, transplanted as neonate only. Cell transplantation conducted in postnatal mice is technically difficult to target to the hippocampus, and as a result, the cells may have been delivered preferentially to the cortex.

In the CA2 and CA3 regions of hippocampus, quantitative image analysis of the NeuN stained area was performed on the sagittal section. The values are reported in Table 3 as the mean total area of NeuN positive cells for each treatment group.

In non-transplanted control PPT1-Scid animals, the area of NeuN positive cells was reduced to 47% of age-matched NOD-Scid mice. In all transplanted groups, there was a significant increase in the area of NeuN positive cells compared to non-transplanted controls. Strikingly, 92% and 97% of NeuN positive cells were detected in the double transplanted-NJ and triple transplanted mice, respectively.

Quantitative image analysis of the area of NeuN positive cells in the cortex was also performed on the sagittal section, and the results are summarized in Table 4. In Table 4, mean values of host neuronal cell survival based on NeuN quantification in the hippocampus are shown. Percentages are normalized against untreated NOD-Scid mice. The mean values reported in Table 4 represent mean total area of ROI. The percentage of NeuN stained area in the defined regions of each transplant group was normalized compared to NOD-Scid (i.e., PPT1+/+) mice.

The area of NeuN staining was 59% in PPT−/− non-transplanted controls compared to NOD-Scid controls. A clear trend regarding increased cell dose transplanted and increased area of NeuN positive staining is present. The triple transplants had significantly more (P<0.05) area of NeuN positive cells compared to non transplanted PPT1-Scid control.

TABLE 4
Mean values of host neuronal cell survival based
on NeuN quantification in the hippocampus.

| | | Not | Group 2 | Group 3 | Group 4 | Group 5 | NOD-Scid |
|---|---|---|---|---|---|---|---|
| Group | | Transplanted | Transplant | Transplant | Transplant | Transplant | Control |
| CA1 | Mean | 2,902 | 10,690 | 7,058 | 12,186 | 21002 | 37,066 |
| | % | 8% | 29%* | 19%* | 33%* | 57%* | 100% |
| CA2/3 | Mean | 17,035 | 31,440 | 23,828 | 33,270 | 34,947 | 36,183 |
| | % | 47% | 87%* | 66%** | 92%* | 97%* | 100% |
| Cortex | Mean | 400,606 | 447,235 | 476,413 | 500,524 | 523,952 | 679,231 |
| | % | 59% | 66% | 70% | 74% | 77%** | 100% |

*P < 0.001,
**P < 0.05 by ANOVA

A systematic analysis of specific brain regions was performed to quantify the numbers of host NeuN⁺ cells in either non-transplanted or HuCNS-SC transplanted PPT1 KO NOD-Scid mice. In all areas examined, more NeuN positive cells were enumerated in the animals that received HuCNS-SC transplants when compared to age-matched non-transplanted controls. Specifically, more cells were detected in the CA1 and CA2/3 regions of the hippocampus and in the cortex of transplanted animals. Moreover, in general, more NeuN positive cells were observed in mice that received the high dose of human cells versus those receiving the lower cell dose. The most striking finding was the observation that up to 57% of host NeuN positive cells survive in the CA1 region of the hippocampus in animals that received the high cell dose compared to only ˜8% of surviving host cells in the non-transplanted group. In addition, in these mice receiving the high cell dose, 97% of normal NeuN levels were detected in CA2 area of the hippocampus.

In this neuroprotection study, control animals were available for the age appropriate range to compare non-transplanted and transplanted groups. The double transplants-NP (Group 3) exhibited slightly less neuroprotection in the CA1 and CA2/3 of the hippocampus, compared to other transplanted groups. This may be the result of the technical difficulty in targeting the hippocampus of pups at the neonatal and postnatal ages. Specifically, stereotactic injection is difficult and the size of the pups vary greatly depending on litter size, the mother's lactation status and how well they are able to compete with siblings for food. Moreover, at P7, the skin is not cut to expose the skull, and the skin is not translucent. Thus, visualizing the blood vessel at the reference lambda point is difficult. In fact only 1 out of 5 double transplant-NP animals had the injection core in the hippocampus, while 6 out of 6 double transplant-NJ animals had the injection core properly targeted to the hippocampus. This emphasizes that the delivery of HuCNS-SCs to the specific target site is important to maximize neuroprotection.

The quantitative NeuN analysis showed that all transplant groups, single, double and triple had high levels of NeuN positive cells compare to the PPT1-Scid non-transplanted controls, with the highest level of neuroprotection observed in the triple transplant group. Transplanted animals have reduced levels of autofluorescence compared to PPT1-Scid controls, which suggest that there is a correlation between neuroprotection and reduced autofluorescence accumulation. Substantial reductions in autofluorescence were observed in the CA1 area, which is concordant with the observation that this is the area exhibiting the highest level of neuroprotection in the transplanted animals.

It is possible that the survival of host neurons might still persist even though lipofuscin levels increase, as long as the HuCNS-SC continue to provide sufficient PPT1 enzyme levels. A working hypothesis is that transplanted HuCNS-SCs provide PPT1 enzyme to host neurons, reduce autofluorescent deposits, and increase their survival. The results of this study demonstrated that transplantation of HuCNS-SCs into the brains of PPT1-Scid mice leads to neuroprotection in the CA1, CA2/3 of the hippocampus and the cortex. Animals receiving the highest cell dose showed the highest level of host neuronal survival. Future studies will address the effect of timing of transplantation, delivery site, and cell dose of HuCNS-SCs on neuroprotection.

### Example 5

### Justification for Clinical Starting Dose

A range of between about 3×10⁶ to about 1×10¹⁰ cells, preferably, between about 5×10⁸ to about 2×10⁹ cells or about 1×10⁸ to about 5×10⁹ cells can be administered to patients with CLN1 or CLN2. Clinical cell dose is based on the toxicology and proof-of-concept pharmacology studies conducted in rodents and non-human primates using a range of cells between about 3×10⁶ to about 1×10¹⁰ cells based on brain weight, and the finding that there were no observed adverse effects. A starting human dose of 500 million cells (the “low dose”) provides about a 1.5-3-fold safety factor relative to the maximal tested dose in the rodents and non-human primates. These doses do not necessarily define the no observed adverse effect level (“NOAEL”) for these species, but, rather, were based on the maximal doses tested. In both mouse and primate safety toxicology studies, there were no observed adverse effects at the maximum dose tested. In proof-of-concept studies, transplanting HuCNS-SC increased PPT1 enzyme level and decreased the accumulation of pathologic autofluorescent lipofuscin material and provided neuroprotection of host neurons in the brains of PPT1 knockout NOD Scid mice (PPT1-KO/NOD-Scid). Based on relative brain weights, the selected doses are anticipated to be within the therapeutic range and to provide an acceptable safety margin.

As there was no toxicity associated with the doses tested in rodents and non-human primates, the choice of clinical starting dose was further guided by the desire to select a putative pharmacologically active dose. A dose range of approximately 0.3-0.8×10⁶ cells in the PPT1-KO/NOD-Scid mouse increased the PPT1 enzyme levels (see FIG. 2), reduced autofluorescent lipofuscin accumulation (see FIG. 3), and neuroprotected host cells (see FIG. 5 and Table 4). The human equivalent to this dose is approximately 500 million cells. Enzyme activity data from patients with neuronal ceroid lipofuscinosis indicates that affliction occurs when PPT1 enzyme activity is less than 2-3% of normal. Thus, the human equivalent dose, as determined by brain weight, for these doses is 360 million and 960 million, respectively.

Therefore, based on toxicology and pharmacology data, a dose of approximately 500 million (the “low dose”) to 1 billion cells (the “high dose”) HuCNS-SC provides an acceptable margin of safety a meaningful increase in PPT1 enzyme level and reduction of accumulated autofluorescent lipofuscin.

We claim:

1. Use of an effective amount of a multipotent self-renewing central nervous system stem cell population in the manufacture of a medicament for treating a lysosomal storage disorder in a mammal.

2. The use of claim 1, wherein the lysosomal storage disorder is characterized by a missing or defective secreted lysosomal enzyme.

3. The use of claim 1, wherein the lysosomal storage disorder is characterized by a mutation in a gene encoding for a secreted lysosomal enzyme.

4. The use of claim 3, wherein the mutation is in the palmitoyl-protein thioesterase 1 (PPT1) gene.

5. The use of claim 3, wherein the mutation is in the tripeptidyl peptidase I (TPP-I) gene.

6. The use of claim 1, wherein the multipotent self-renewing central nervous system neural stem cell population is obtained from a human.

7. The use of claim 1, wherein the cells of the multipotent CNS neural stem cell population have been proliferated in a suspension culture.

8. The use of claim 1, wherein the cells of the multipotent CNS neural stem cell population have been proliferated in an adherent culture.

9. The use of claim 6, wherein the lysosomal storage disorder is a neuronal ceroid lipofuscinoses.

10. The use of claim 9, wherein the neuronal ceroid lipofuscinoses is selected from the group consisting of infantile NCL and late infantile NCL.

11. The use of claim 1, wherein the medicament is suitable for transplantation to the CNS of a mammal.

12. The use of claim 11, wherein the mammal is a human.

13. The use of claim 12, wherein the medicament is suitable for transplantation into the hippocampus.

14. The use of claim 12, wherein the medicament is suitable for transplantation into the cortex.

15. The use of claim 11, wherein the medicament is suitable for transplantation by subcortical injection or by intraventricular injection.

16. The use of claim 11, wherein the effective amount comprises between 3×10⁶ and 1×10¹⁰ cells.

17. The use of claim 11, wherein the effective amount comprises between 1×10⁸ and 5×10⁹ cells.

18. The use of claim 11, wherein the medicament is administered to the mammal in one dose.

19. The use of claim 11, wherein the medicament is administered to the mammal in multiple doses.

20. The use of claim 1, wherein the effective amount of the multipotent CNS neural stem cell population is obtained from the mammal's neural tissue.

21. The use of claim 1, wherein the effective amount of the multipotent CNS neural stem cell population is derived from neonatal, juvenile, or adult mammalian neural tissue.

22. Use of an effective amount of a multipotent self-renewing CNS neural stem cell population in the manufacture of a medicament for reversing or slowing neurodegeneration in a patient suffering from or at risk for developing a neuronal ceroid lipofuscinoses, wherein the medicament is suitable for transplantation into the hippocampus, the cortex, or both of the patient.

23. The use of claim 22, wherein the effective amount comprises between 3×10⁶ and 1×10¹⁰ cells.

24. The use of claim 22, wherein the effective amount comprises between 1×10⁸ and 5×10⁹ cells.

25. The use of claim 22, wherein the neuronal ceroid lipofuscinoses is selected from the group consisting of infantile NCL and late infantile NCL.

26. The use of claim 22, wherein the transplantation occurs by subcortical injection or by intraventricular injection.

27. The use of claim 22, wherein the medicament is transplanted into the patient in one dose.

28. The use of claim 22, wherein the medicament is transplanted into the patient in multiple doses.

29. The use of claim 22, wherein the effective amount of the multipotent CNS neural stem cell population is obtained from the mammal's neural tissue.

30. The use of claim 22, wherein the effective amount of the multipotent CNS neural stem cell population is derived from neonatal, juvenile, or adult mammalian neural tissue.

31. A pharmaceutical composition for treating a lysosomal storage disorder, said composition comprising between 3×10⁶ and 1×10¹⁰ cells.

32. A pharmaceutical composition for treating a lysosomal storage disorder, said composition comprising between 1×10⁸ and 5×10⁹ cells

33. A kit comprising in one or more containers, the pharmaceutical composition of claim 31.

34. A kit comprising in one or more containers, the pharmaceutical composition of claim 32.
